(19) United States
(12) Patent Application Publication
(10) Pub. No.: US 2026/0090510 A1
(43) Pub. Date: Apr. 2, 2026

(54) USE OF RESIN ACIDS AS DEFENSE GENE INDUCERS FOR BIOTIC STRESS IN PLANTS

(71) Applicant: Action Pin, Dax (FR)

(72) Inventors: Camille HUET, Dax (FR); Gérard MOLLA, Dax (FR)

(21) Appl. No.: 19/113,076

(22) PCT Filed: Sep. 26, 2023
(86) PCT No.: PCT/EP2023/076605

§ 371 Date: Mar. 19, 2025

(30) Foreign Application Priority Data
Sep. 27, 2022 (FR) FR2209793
Sep. 29, 2022 (EP) 22198819.9

(51) Int. Cl. A01H 1/00

(52) U.S. Cl. CPC A01H 1/1255

(57) ABSTRACT

Disclosed herein is a method of using resin acids as defense gene inducers for biotic stress in a plant. Also disclosed herein are a composition including rosin and a method of using the composition as a defense gene inducer for biotic stress in a plant.

### FIELD OF THE INVENTION

The present invention concerns the use of resin acids as defense gene inducers in a plant.

The present invention also relates to a composition comprising rosin and the use thereof as a defense gene inducer for biotic stress in a plant.

### PRIOR ART

Nowadays, there is a growing concern about environment with a goal to protect our planet, especially in agriculture. However, pest invasion in crops is huge and results in high economic loss for the agricultural sector. In particular, pests such as fungi and/or oomycetes are viral in grapevines. If left untreated, grape downy mildew (GDM) attacks can cause yield losses and reduce grape quality.

Common solutions to keep damages at minimum during the growing season involve repeated spraying of chemical pesticides on crops.

Among these pesticides, copper-based fungicide compositions are commonly used in conventional, integrated and organic agriculture to control devastating plant diseases such as grapevine downy mildew, potato and tomato late blight, apple scab, and a wide range of other plant pathogens. In particular, the Bordeaux mixture is commonly used as control agent against grapevine downy mildew. The latter is caused by the fungus or oomycete Plasmopara viticola.

Since 2006, the European Commission regulation has limited the use of copper as it is environmentally toxic. Despite its unfavorable eco-toxicological profile, the use of copper is still tolerated due to its unique properties as a wide-spectrum fungicide and bactericide especially against grapevine downy mildew.

This strategy keeps damages levels low but has major environmental consequences, high production costs, risks to human health, particularly for vine growers. Furthermore, these substances may adversely impact the winemaking processes and as such wine quality.

Copper-based fungicides are used to control the disease in organic vineyards Control of pathogens by means of plant-derived plant protection products can be an effective, sustainable, and environmentally friendly method for pest management in integrated pest management (IPM) and organic farming systems. Natural organic compounds are often easily degraded in a natural environment, e.g. by degradation by UV-light, and are thus less likely to accumulate in the environment or to cause residues on food.

Recent studies have demonstrated the fungicidal properties of resinic acid derivatives as well as phenolic compounds such stilbenes, flavonoids and lignans. In particular, Savluchinske-Feio and al. (1) revealed that the antimicrobial activity of resinic acid derivatives is expressed via killing of microorganism and/or limiting their growth. This study concentrates on disclosing the inhibitory concentration IC50 or IC100 of different derivatives but does not mention the activity of resin acids on defense genes.

Gabaston et al. (2, 3) have shown that stilbene-enriched extracts from Vitis vinifera waste (cane, wood and root) and common spruce bark are highly efficient against grapevine downy mildew caused by the fungus or oomycete Plasmopara viticola.

Alternative solutions to protect crop from disease has relied on the use of elicitors that enhance the resistance of the plant to a pathogen. Elicitors are natural or synthetic compounds that induce a defense response in plants as induced by a pathogen infection. They stimulate the activity of the plant defenses. Elicitors are non-specific and differ widely in their chemical nature including protein, oligosaccharides, glycoprotein and lipids.

Therefore, there is a need for providing non-toxic, efficient, cost-effective and ecologically friendly phytosanitary products to protect crop from fungal damage.

Particularly, there is a high interest in providing alternative or complementary compositions to copper-based fungicides and that allow reducing the use of copper for preventing or treating disease caused by fungi and/or oomycetes in plants.

### SUMMARY OF THE INVENTION

Herein disclosed, the use of resin acids as defense gene inducers for abiotic and/or biotic stress in a plant. Particularly, the present invention concerns the use of resin acids as defense gene inducers for biotic stress in a plant.

The invention further concerns a composition comprising rosin and a solvent.

Herein disclosed the use of resin acids as a biostimulant or as a phytosanitary product. Particularly, the present invention also relates to the use of resin acids as a phytosanitary product.

Herein disclosed, the use of a composition comprising rosin as a defense gene inducer for abiotic and/or biotic stress in a plant. Particularly, the invention also relates to the use of a composition comprising rosin as a defense gene inducer for biotic stress in a plant.

The present disclosure further provides a method for the treatment or prevention of a disease caused by at least one fungus and/or oomycete in a plant, said method comprising a step (i) of applying to the plant a composition comprising rosin.

### DESCRIPTION OF THE FIGURES

FIG. 1 illustrates a summary of the associated proteins and pathways identified to explain the effects of the rosin composition according to the invention on vine leaves.

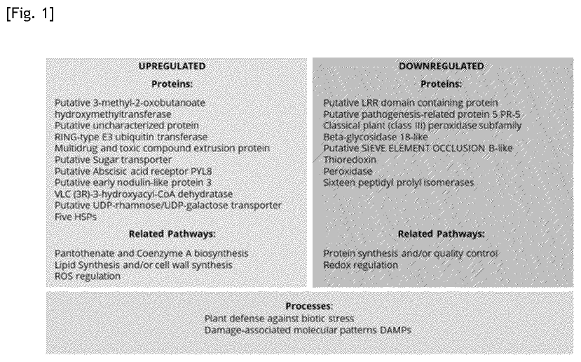

### DEFINITIONS

For the purposes of the present invention, “defense gene inducer” refers to the induction of the expression of genes representative of the main defense pathways in a plant, from defense proteins (pathogenesis related proteins) to signaling pathways (salicylic acid, jasmonic acid and ethylene).

For the purposes of the present invention, “pine stump” means the remaining portion of the trunk with the roots still in the ground after a pine tree has been cut and felled.

For the purposes of the present invention, “biostimulant” means a substance that stimulates plant nutritional processes independently of the nutrients it contains with the aim of improving one or more of the following characteristics of plants or their rhizosphere: nutrient use efficiency, abiotic stress tolerance, quality characteristics, availability of nutrients confined in the soil or rhizosphere (according to EU Regulation 2019/1009).

Biostimulants can be certain natural preparations of little concern (PNPP). The PNPP are:
either natural substances for biostimulant use (NSPB) or
basic substances.

The basic substances are defined by Article 23 of Regulation (EC) 1107/2009. They are substances with phytosanitary interest but whose main use is other than plant protection (e.g. foodstuffs).

Within the context of the present invention, the term “biotic stress” designates a stress that occurs as a result of damage done to plants by living organism such as plant pathogens. The term “pathogens” designates all pathogens of plants in general such as bacteria, viruses, fungi, parasites or insects. More preferably the pathogens are fungal pathogens such as fungi and/or oomycetes.

Within the context of the present invention, the term “abiotic stress” designates a stress that occurs as a result of damage done to plants by non-living environmental factors such as drought, extreme cold or heat, high winds, salinity, heavy metals.

### DETAILED DESCRIPTION OF THE INVENTION

The invention concerns the use of resin acids as defense gene inducers for biotic stress in a plant.

The present invention also relates to the use of resin acids as a phytosanitary product.

In particular, the resin acids as disclosed herein have an activity against at least one fungus and/or oomycete.

Surprisingly, the Applicant has discovered that resin acids induce plant's defense and can be used as defense gene inducers for biotic stress against pathogens.

Thus, the resin acids can be classified in the category of stimulating or eliciting molecules. Examples of the invention show that using resin acids derived from rosin or comprised in rosin allow a reduction of the attack of fungi and/or oomycetes, in particular the attack of mildew grapevines (severity and frequency) on the trials carried out in fields. Advantageously, compositions comprising rosin are particularly effective for the prevention of fungal damage and for the treatment of fungal diseases in plants.

Remarkably, the association of a composition as disclosed herein with a copper-based composition in low content is as efficient as traditionally used copper-based fungicides.

Therefore, the fungicidal composition based on resin acids derived from rosin or comprised in rosin as disclosed herein allows reducing the amount of copper-based fungicide used in vineyards.

### Resin Acids

The present invention relates to resin acids and their capacities to induce plant's defense. The term “resin acids” is understood as any compounds or mixtures obtained or derived from wood and comprising one carboxylic acid functional group and a fused tricyclic group. Said resin acids are tricyclic diterpene monocarboxylic acids or are derived from tricyclic diterpene monocarboxylic acids. The resin acids comprise acids from abietic-type acids, from primaric-type acids and other acids such as communic acid or mercusic acid. In particular, the present invention relates to resin acids from abietic-type acids and their capacities to induce plant's defense.

Examples of resin acids include, but are not limited to, abietic acid, levopimaric acid, palustric acid, neoabietic acid, dehydroabietic acid, isopimaric acid, pimaric acid, levopimaric acid, sandaracopimaric acid, communic acid, oxidized resinic acid, dimerized resin acids, polymerized resin acids or a mixture thereof.

According to any embodiments of the invention, the resin acids is in a form of a mixture comprising at least one acid selected from the group of dehydroabietic acid, abietic acid, primaric acid, dimerized resin acids, polymerized resin acids and a mixture thereof.

Particularly, the resin acids is in a form of a mixture comprising at least one acid selected from the group of dehydroabietic acid, dimerized resin acids, polymerized resin acids and a mixture thereof.

The resin acids are preferably derived from rosin or are comprised in rosin.

### Rosin

Rosin, also called colophony, is typically obtained from conifers, in particular from pine trees.

Rosin is a form of resin and is solid at room temperature.

It is typically produced by heating liquid resins to vaporize the volatile liquid terpene components.

Rosin mainly consists of resin acids such as cited above.

Rosin's softening point and acid value number are key quality parameters.

The softening point may be determined by measuring the glass transition temperature of rosin. The softening point of a rosin is determined according to well-known methods to the person skilled in the art, in particular by following the procedures described in patent WO2022/133426.

The rosin according to the invention has typically a softening point comprised from 50° C. to 175° C., preferably from 60° C. to 140° C., preferably from 75° C. to 125° C., more preferably from 80° C. to 120° C.

Depending on its application, rosin may require to be softened, for example when used in consumer goods or to be deposited. The value of the softening point will impact the properties of the rosin.

Also called “acid value”, the acid number is used to quantify the amount of acid present in rosin. The acid number of a rosin is determined according to well-known methods to the person skilled in the art, in particular by following the procedures described in patent WO2022/133426.

This acid number may be less than about 175, e.g., from about 50 to about 175, preferably from 90 to 170, preferably from 100 to 170 or from 90 to 140, preferably from 110 to 170 or from 90 to 130, preferably from 120 to 170 or from 90 to 130, preferably from 130 to 170 or from 90 to 130, preferably from 140 to 170 or from 90 to 130, even more preferably from 150 to 170 or from 90 to 130.

Rosin is a solid form of pine oleoresin from pine trees obtained by incising the bark of the tree. Distillation of the oleoresin leads to the production of a volatile fraction mainly composed of terpenes and a non-volatile fraction in a solid form that corresponds to rosin.

Rosin may be solid at room temperature, semi-transparent and varies in color from faint yellow to a darker brown color, or even black.

Suitable sources of rosin include gum rosin, wood rosin, tall oil rosin, derivatives thereof or a mixture thereof.

A preferred source is wood rosin, gum rosin, derivatives thereof.

Gum rosin may be derived from a resin extrudate of a tree or other plant and may be harvested by tapping or wounding the tree and then collecting and processing the extrudate. The tall oil rosin is a byproduct of the distillation of crude tall oil during the Kraft process of wood pulp manufacture when pulping pine trees.

Wood rosin may be derived from materials that are harvested from pine tree, particularly from pine tree stumps, for example through solvent extraction and/or distillation.

Advantageously, these resins are sticky, resistant to rain and therefore allow for a more efficient induction.

According to a particular embodiment, a preferred rosin is gum rosin, particularly a derivatives gum rosin. By the term “a derivatives gum rosin”, it is understood gum rosin which is chemically modified; e.g. the gum rosin may be further hydrogenated, dehydrogenated, dismutated, esterified, dimerized, polymerized. Particularly, a preferred rosin is a dismutated gum rosin or a dimerized gum rosin. The person skilled in the art is well aware of conditions to apply to derivative gum rosin.

According to another particular embodiment, a preferred rosin is wood rosin.

In some embodiments, the rosin is wood rosin extracted from pine, especially from pine stump.

In some embodiments, the rosin is wood rosin extracted from pine provided that the wood rosin is not extracted from knot.

Advantageously, these resins are sticky, resistant to rain and therefore allow for a more efficient induction.

According to any embodiment of the invention, the rosin is a wood rosin extracted from pine or is a dismutated gum rosin or a dimerized gum rosin and the rosin comprises dehydroabietic acid (CAS No. 1740-19-8). The dismutated gum rosin is a gum rosin treated with a catalyst such as Palladium in metallic form, Pd/C, platinum in metallic form, Pt/C, nickel in metallic form, Ni/C or iodine. The dismutation is also known as disproportionation. The dimerized gum rosin is a gum rosin treated with Bronsted acid such as sulfuric acid or formic acid or Lewis acids such as BCl₃, BF₃ or ZnCl₂.

In some embodiments, the Applicant also demonstrated that wood rosin marketed for example as Vinsol® MC, Vinsol® and Belro® is able to induce grapevines defense genes for biotic stress.

Vinsol®, Vinsol® MC and Belro® are three resins produced by Pinova from pine stump extraction, registered under Reach regulation with the same CAS number as wood rosin; i.e. 8050-09-7.

Wood rosin comprises resin acids such as described above that have been shown in the present invention to induce plant's defense gene for biotic stress.

Suitable rosin includes any rosin registered under Reach regulation with CAS number 8050-09-7 corresponding to wood rosin or CAS number 65997-05-9 corresponding to polymerized rosin. Non-limiting examples of commercially available rosin includes, but are not limited to, Vinsol®, Vinsol MC® and Belro® commercialized by Pinova or Resigral 52®, Polygral® 140 and Polygral® 95 commercialized by DRT.

Typically, the rosin is obtained from pine of the genus Pinus. The pine tree is preferably selected from the following species:
Aleppo pine or Jerusalem pine (Pinus halepensis)
Calabrian pine or Turkish pine or Persian pine (Pinus brutia)
Macedonian pine or Balkan pine (Pinus peuce)
Maritime pine or Landes pine (Pinus pinaster)
Mountain pine or mugo pine, Pinus mugo Black pine (Pinus nigra)
Pinus pinea Scots pine (Pinus sylvestris)
Pinus contorta
Pinus sibirica
Monterey pine (Pinus radiata)
Frankincense pine (Pinus taeda)
Jack pine (Pinus banksiana)
Caribbean pine (Pinus caribaea)
Pinus tecunumanii
Ponderosa pine (Pinus ponderosa)
Slash pine (Pinus elliottii)
Bosnian pine (Pinus heldreichii, syn. Pinus leucodermis)
Canary Island pine (Pinus canariensis)
Cembro pine, arolle, alvier, (Pinus cembra)
Longleaf pine (Pinus Palustris)

Preferably the rosin is obtained from pine of the species Pinus palustris, Pinus pinaster and Pinus elliottii.

According to a particular embodiment, the wood rosin is extracted from pine stump of the genus Pinus. Particularly, the wood rosin is extracted from pine stump of the species Pinus palustris and Pinus elliottii.

The wood rosin is typically obtained from pine stump by classical extraction processes. For example, wood rosin can be obtained by extraction with hot water, by extraction with subcritical water, by extraction with a water/organic solvent mixture, by extraction with a hydroalcoholic solvent mixture, by extraction with at least one organic solvent.

Preferably, said extract is obtained by extraction with a water/organic solvent mixture or by extraction with at least one organic solvent.

Any organic solvent mixture current in such extraction process can be used for the purposes of the invention.

Non-limiting examples include hydrocarbon solvents such as hexane, cyclohexane, heptane, octane or ether-based solvents as methyl tetrahydrofuran, alcoholic solvents such as methanol, ethanol, propanol, 2-methylbutan-2-ol or mixtures thereof, ester-based solvents such as n-butyl acetate, iso-propyl acetate, ethyl acetate or ketone solvent such as acetone, acetophenone, butanone, cyclopentanone or mixtures thereof.

In some embodiments, the rosin comprises from 15 to 95 wt. % of resin acids with respect to the total weight of rosin, particularly from 20 to 85 wt. %, particularly from 40 to 80 wt. %, particularly from 60 to 80 wt. % or from 40 to 75 wt. %, more preferably from 65 to 75 wt. % with respect to the total weight of rosin. In a particular embodiment, the rosin comprises ate least 85 wt. % of resin acids with respect to the total weight of rosin.

In some embodiments, the rosin comprises at least 30 wt. % of dehydroabietic acid with respect to the total weight of rosin, particularly at least 40 wt. %, particularly at least 45 wt. %, more preferably at least 50 wt. % with respect to the total weight of rosin.

In some embodiments, the rosin comprises at least 20 wt. % of dimerized resin acids with respect to the total weight of rosin, particularly at least 30 wt. %, more particularly at least 35 wt. % with respect to the total weight of rosin.

The rosin as disclosed herein may further comprise flavonoids, stilbenes, dimethoxystilbene, wax, polymerized terpenes, lignans and lignan degradation products such as phenolic derivatives.

Examples of flavonoids include pinocembrin.

Typically, the stilbenes are selected in the group consisting in pinosylvin monomethyl ether and pinosylvin dimethyl ether.

Examples of lignans or lignan degradation products include nortrachelogenin, phlobaphenes, carboxylated phlobaphenes, lignin hydroxylactone.

Preferably, the rosin is wood rosin extracted from pine stump or is a dismutated gum rosin or a dimerized gum rosin which is used as a defense gene inducer for biotic stress in a plant.

In some embodiments, the plant is a crop plant.

Non limitative examples of plant include grapevine, vegetables such as a tomato plant, a potato plant, lettuce, a cucurbits plant such as melon and cereals such as wheat, barley, oat, rye.

Preferably, the plant is selected in the group consisting in grapevine and cereals.

In some embodiments, the defense gene is a gene encoding an enzyme in the phenylpropanoid pathway or pathogenis-related protein.

More specifically, the rosin is useful for enhancing defence genes selected from pathogenesis-related proteins, phenylpropanoids, isoprenoids, oxidative stress, parietal modification and signalling pathways such as salicylic acid, jasmonic acid and ethylene.

Plants are exposed to biotic stresses such as infection by pathogens, but also to abiotic stresses such as cold, heat, drought and salt. Adaptive strategies are put in place by plants to avoid or mitigate these adverse effects. Fatty acids and reactive oxygen species (ROS) appear as general defenders and play a key role in the acclimatisation process of plants to abiotic and biotic stresses. Indeed, the synthesis of fatty acids will be induced by the plant to thicken its cuticle and thus protect itself from dehydration or pathogen attack. Similarly, the synthesis of ROS will be induced by the plant to cope with UVs or a fungal attack.

As demonstrated in the examples, proteomic analysis experiments on vine leaves treated with composition comprising wood rosin as disclosed herein showed a DAMP (damage-associated molecule pattern) response induction, including proteins that may be involved in cell wall synthesis, secondary metabolite and sugar transport, xenobiotic detoxification, abscisic acid receptors, and reactive oxygene species regulation.

Thus the related pathways involved in using the present invention include the pantothenate and coenzyme A biosynthesis, the lipid synthesis and/or cell wall synthesis and the reactive oxygene species regulation.

In some embodiments, the rosin may be used in association with a fungicide.

This association is of particular interest as an alternative to the sole use of a copper-base fungicide treatment.

Furthermore, this association is as efficient as traditional copper-based fungicide when sprayed on a plant.

Preferably, the fungicide is selected from the group consisting of a copper-based product such as copper hydroxide, Bordeaux mixture, dithiocarbamate, dicarboximide, potassium phosphonate, dipotassium phosphonate, monopotassium phosphite, disodium phosphonate, carboxylic acid amide, cyanoimidazole, an acetamide, a phenylamide and a benzamide, phthalimide, cyanoacetamide oxime, sulfamoyl triazole, triazolo pyrimidylamine and piperidinyl triazole isoxazolines.

In some preferred embodiments, the copper-based fungicide is copper hydroxide. This fungicide is marketed under the brand name Heliocuivre® by Action Pin.

### Rosin Composition

The present invention also relates to a composition comprising rosin as described above and a solvent and the use thereof as a defense gene inducer for biotic stress in a plant.

In some embodiments, the solvent is selected from the group consisting of dimethysulfoxide (DMSO), fatty acid methyl ester, N,N-Dimethyldecanamide such as dimethylamide of C10 fatty acid marketed under the brand name Genagen 4296®, 1-butylpyrrolidin-2-one marketed under the brand name Genagen NBP®, alkyl-1,2-diol with the alkyl chain having from 5 to 10 carbon atom, 2,5,7,10-tetraoxaundecane, butylal, ethylal, 2-ethylhexylal and ethyl lactate.

Typically, the composition comprises from 35 to 60 wt. % of solvent with respect to the total weight of composition, preferably from 35 to 40 wt. %, more preferably from 36 to 40 wt. % of solvent with respect to the total weight of composition.

The composition according to the present invention may further comprises an emulsifying agent, a penetrating agent and a wetting agent.

In some embodiments, the emulsifying agent is selected from the group consisting of ethoxylated castor oil, ethoxylated fatty alcohols, alkyl polyglucosides, ethoxylated rapeseed oil, glycereth-2-cocoate, polyethoxylated polyol ester such as Levenol C201®, polyoxyethylene sorbitan monolaurate also called polysorbate or Tween 20.

Typically, the composition comprises from 15 to 40 wt. % of emulsifying agent with respect to the total weight of composition, preferably from 20 and 30 wt. %, preferably from 22 to 28 wt. %, more preferably from 23 to 27 wt. % of emulsifying agent with respect to the total weight of composition.

The composition may further comprise a penetrating agent selected from the group consisting of tall oil fatty acids, unsaturated fatty acids such as oleic acid or linoleic acid, and saturated fatty acids such as palmitic acid and stearic acid.

In general, the composition comprises from 1 to 15 wt. % of penetrating agent with respect to the total weight of composition, preferably from 5 and 13 wt. %, preferably from 7 to 12 wt. %, more preferably from 8 to 11 wt. % of penetrating agent with respect to the total weight of composition.

In some embodiments, the wetting agent is a terpene alcohol, in particular the wetting agent is selected from the group consisting of 2,6-dimethyloct-7-en-2-ol, alpha terpineol, 4(8)-p-menthen-1-ol, cis-3,7-dimethyl-2,6-octadien-8-ol, trans-3,7-dimethyl-2,6-octadien-1-ol, 10-hydroxymethylene-2-pinene, 3,7-dimethyloct-6-en-1-ol.

Advantageously, the use of terpene allows improving the spreading of the composition according to the invention on a plant.

The composition comprises typically from 5 to 30 wt. % of wetting agent with respect to the total weight of composition, preferably from 5 to 20 wt. %, preferably from 6 and 15 wt. %, preferably from 7 to 12 wt. %, more preferably from 8 to 11 wt. % of wetting agent with respect to the total weight of composition.

In some embodiments, the composition comprises from 15 to 25 wt. % of rosin with respect to the total weight of composition, preferably from 16 to 23 wt. %, preferably from 16 to 20 wt. % of rosin with respect to the total weight of composition. In some embodiments, the composition comprises 17 wt. % of rosin with respect to the total weight of composition.

In some preferred embodiments, the rosin is wood rosin extracted from pine stump or is a dismutated gum rosin or a dimerized gum rosin.

The present invention provides typically a composition comprising from 35 to 40 wt. % of solvent with respect to the total weight of composition, from 20 to 30 wt. % of emulsifying agent with respect to the total weight of composition, from 15 to 25 wt. % of rosin with respect to the total weight of composition, from 1 to 15 wt. % of penetrating agent with respect to the total weight of composition and from 5 to 30 wt. % of wetting agent with respect to the total weight of composition. In some preferred embodiments, the rosin is wood rosin extracted from pine stump or is a dismutated gum rosin or a dimerized gum rosin.

Advantageously, the composition according to the invention increases the plant's defense against fungi and/or oomycetes.

The composition of the invention may be in solid or liquid form. Preferably it is in liquid form.

In some embodiments, the composition as disclosed herein is in a form of an emulsifiable concentrate.

In some embodiments, the composition comprising rosin is provided in a form suitable for application by spraying.

Advantageously, the composition according to the present invention allows reducing the dose of a copper fungicide composition used on a plant.

In some embodiments, the dose of copper is reduced from 20 to 50% with respect to commonly used dose on a plant.

The composition may be prepared by conventional means, e.g. by mixing the different components.

The invention also relates to the use of a composition as disclosed herein as a phytosanitary product.

### Plant Protection Composition

The invention also relates to a plant protection composition comprising a rosin-based composition as disclosed above and a fungicide.

Preferably, the fungicide is selected from the group consisting of a copper-based product such as copper hydroxide, Bordeaux mixture, dithiocarbamate, dicarboximide, potassium phosphonate, dipotassium phosphonate, monopotassium phosphite, disodium phosphonate, cyanoimidazole, a carboxylic acid amide, an acetamide, a phenylamide and a benzamide, phthalimide, cyanoacetamide oxime, sulfamoyl triazole, triazolo pyrimidylamine and piperidinyl triazole isoxazolines.

In some preferred embodiments, the copper-based fungicide is copper hydroxide. This fungicide is marketed under the brand name Heliocuivre® by Action Pin.

In some preferred embodiments, the rosin is wood rosin extracted from pine stump or is a dismutated gum rosin or a dimerized gum rosin.

### Method

The present invention also relates to a method for the treatment or prevention of a disease caused by at least one fungus and/or oomycete in a plant, said method comprising a step (i) of applying to the plant a composition according to the present invention.

In some embodiments, the method comprises a step (i) of applying to the plant a composition in an effective amount according to the present invention.

The present invention allows enhancing disease resistance to treated plant.

The present invention also concerns a method for inducing defense gene for biotic stress in a plant using resin acids.

The present invention also relates to a method for inducing defense gene for biotic stress in a plant using rosin according to the invention.

In some embodiments, the present invention also relates to a method for inducing defense gene for biotic stress in a plant using a composition according to the invention.

In some embodiments, the method according to the present invention may further comprises a step (ii) of applying a fungicidal composition to the plant.

Preferably, the fungicidal composition comprises copper.

Advantageously, the association of the composition as disclosed herein and a copper-based fungicide allows reducing the amount of copper used on a plant and shows a similar activity to prevent or treat disease caused by fungi and/or oomycetes.

Steps (i) and (ii) may be concomitant or sequential in one order or another.

Preferably, the disease caused by at least one fungus and/or oomycete is selected from the group consisting of grapevine downy mildew, grapevine powdery mildew, grapevine botrytis, potato and tomato late blight, apple scab and wheat rust.

Even more preferred disease is grapevine downy mildew, grapevine powdery mildew, grapevine botrytis or wheat rust.

Examples of fungus and/or oomycetes include, but are not limited to, Plasmopara viticola, Phytophthora infestans, Pseudoperonospora cubensis, Bremia lactucae, Botrytis cinerea, Erysiphe necator, Uromyces sp. and Puccinia sp.

Preferably, the fungus and/or oomycete is Plasmopara viticola that causes grapevine downy mildew.

In some embodiments, the composition according to the present invention is applied from 0.5 to 2 L per hectare per leaf wall area, preferably 1 L per hectare per leaf wall area.

In some embodiments, the composition according to the present invention is applied from 0.5 to 3 L per hectare, preferably from 0.8 to 2.4 L per hectare.

In terms of application unit, 0.5 liter per hectare per leaf wall area is equivalent to 0.8 liter per hectare.

The dose is depending on the leaf wall area of the plant and the leaf wall area depends on the plant inter-row width and height.

In the present invention, the leaf wall area (LWA) was calculated according to the following formula:

$$LWA = ( 2 \times H \times 10 \; 000 ) / D$$

wherein LWA is the leaf wall area (m²/ha),
H is the height of the leaf wall (m) measured vertically between the lowest leaf and highest leaf of the wall area and
D is the distance measured between the plant's rows (m)

When the pressure pest is high the rosin-based composition as disclosed herein may be used in combination with a fungicidal composition.

The method typically comprises applying from 0.5 to 3 L per hectare of a rosin-based composition as disclosed herein and from 0.5 to 2 L per hectare of a fungicidal composition.

In some embodiments, the fungicidal composition is applied from 0.5 to 2 L per hectare, preferably from 0.5 to 1 L per hectare, even more preferably from 0.5 to 0.75 L per hectare. In some preferred embodiments, the fungicidal composition is applied at 0.5 L per hectare.

In some embodiments, the fungicidal composition is a copper-based composition and is applied from 0.5 L to 2 L per hectare which corresponds to a copper dose from 200 to 800 g per hectare of copper.

In general, the composition according to the present invention is administered by means of spraying on a plant.

In some preferred embodiments, the plant is a crop plant.

Spraying may be repeated several times to the plant.

In some embodiments, spraying may be repeated at least twice, preferably between 2 and 10 times.

In some embodiments, the spraying may be repeated at least twice, 4 to 30 days apart, preferably 7 to 20 days apart.

Non limitative examples of plant include grapevine, vegetables such as tomato plant, potato plant, lettuce, cucurbits plants such as melon and cereals such as wheat.

Preferably, the plant is grapevine.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### BIBLIOGRAPHIC REFERENCES

(1) Savluchinske-Feio et al., Applied Microbiol and Biotechnology, 2006, 72, 430-436 “Antimicrobial activity of resin acid derivatives”.
(2) Gabaston, J. et al., Industrial Crops and Products, 2017, 103, 267-273 “Stilbenes from common spruce (Picea abies) bark as natural antifungal agent against downy mildew (Plasmopara viticola)”.
(3) Gabaston, J. et al., International Viticulture and Enology Society, 2022, 56, 55-68 “A grapevine by-product extract enriched in oligomerised stilbenes to control downy mildews: focus on its modes of action towards Plasmopara viticola”.

### EXAMPLES

### Materials and Methods

Wood rosin is obtained from commercial products Vinsol® and Vinsol® MC that are two resins from pine stump produced by Pinova, registered under Reach regulation with the same CAS number as wood rosin that is CAS 8050-09-7.

### Example 1: Preparation of Compositions According to the Invention

First, dimethyl sulfoxide was mixed with a wetting agent as described here-above. The resulting mixture was then stirred and heated at 50° C.

Wood rosin tablets was gradually added to the previous mixture under strong stirring.

The crude mixture was left to stir until complete solubilization of the wood rosin. The stirring was then slowed down and the ethoxylated castor oil and tall oil fatty acids were added.

The mixture was finally stirred for a further 20 min to give a homogeneous mixture.

Emulsifiable concentrates C1 from Vinsol® MC or C2 from Vinsol® are obtained according to this process.

### Example 2: Evaluation of the Gene Defense Inducing Activity of a Composition C1 Prepared According to Example 1 in Grapevine

The capacity to induce plant defense genes in grapevine model was evaluated with qPFD platform method with 15 markers genes (INRA patent WO2011/161388); Grape variety: Cabernet Sauvignon.

The 15 marker genes were selected from pathogenesis related proteins, phenylpropanoids and oxidative stress.

RNAs were extracted from leaf tissue, which were treated with 6 different compositions.

Yield and quality of extracted RNA were assessed with a spectrophotometer. Gene expression levels were calculated and normalized.

The results are shown in Table 1.

In this Table, the following six compositions were assessed and compared to the inducer control at two days (D2) after treatment at day 0 (D0). The six compositions are as follows:
Emulsifiable concentrate from Vinsol® MC, pine stump extract: composition C1
Emulsifiable concentrate from Vinsol®, pine stump extract: composition C2
Emulsifiable concentrate from pine knot extract: composition PK1
lignanes fraction: F1
mixture of stilbenes and flavonoids: F2
resin acids fraction: F3

Fractions F1 to F3 were obtained by purification of a crude resin extract by silica chromatography.

TABLE 1
Heatmap of relative quantitative expression of 15 defenses genes from the qPFD ® tool in grapevine.
Assessment of six different candidate products, compared to an inducer control, at two days after day treatment.

| | PR proteins | | | | | | | phenylpropanoids | | | | | Oxidative stress | | |
|---|---|---|---|---|---|---|---|---|---|---|---|---|---|---|---|
| Modalities | PR1 | PR2 | PR4 | PR5 | PR8 | PR14 | PR15 | PAL | CHS | DFR | ANS | PPO | APOX | GST | POX |
| Water | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| Inducer control | 1.5 | 0.8 | 1.7 | 1.0 | 0.7 | −1.1 | 1.6 | 0.6 | 0.0 | 0.6 | 0.3 | 1.3 | 0.6 | 5.5 | −1.0 |
| PK1 | 0.6 | −0.1 | 1.2 | −0.5 | −0.1 | −0.3 | 0.1 | 0.2 | −0.8 | 0.2 | −0.2 | 0.5 | −0.2 | 5.6 | −0.9 |
| C1 | 1.6 | 1.2 | 1.7 | 1.1 | 0.7 | −0.3 | 1.8 | 1.3 | 0.4 | 1.0 | 0.8 | 1.1 | 0.2 | 6.4 | −0.2 |
| C2 | 2.0 | 1.2 | 1.4 | 0.6 | 0.5 | −0.2 | 1.9 | 1.9 | −1.0 | 0.0 | −0.4 | 1.6 | 0.2 | 4.4 | −0.1 |
| F1 | 2.2 | −0.1 | 2.0 | 0.4 | 0.5 | 0.6 | 0.4 | −1.0 | −1.1 | −0.2 | −0.1 | −0.2 | −0.2 | 4.4 | 0.0 |
| F2 | 3.7 | 2.5 | 3.7 | 2.3 | 2.0 | −0.1 | 6.4 | 1.7 | 0.4 | 1.0 | 0.8 | 2.9 | 0.3 | 7.5 | −0.2 |
| F3 | 5.1 | 2.9 | 4.7 | 3.0 | 2.0 | 0.3 | 6.5 | 1.6 | 1.0 | 1.3 | 1.0 | 2.7 | 0.4 | 8.2 | −0.4 |

A H₂O₂ application is performed 24 h (at one day) post-treatment in order to mimic a pathogen attack. The average relative gene expressions is obtained by RT-qPXR in two independent biological repetitions and relative to the water control.

The qPFD analysis proves that wood rosin and more precisely resin acids induce grapevine defense genes for biotic stress.

All the trials in the vineyard carried out over more than 2 years show that the rosin Vinsol® and Vinsol® MC composed of resin acids, and formulated in an emulsifiable concentrate, has an activity against mildew. The defense gene expression is higher when using wood rosin from stump pine extract as compared to a pine knot extract.

F3 comprising only resin acids provides the best results confirming their activity as defense gene inducers for biotic stress.

This efficiency is due to the fact that the resin acids present in the rosin, induce the genes of defenses of the vine. The stimulated plant can set up a defense system against the mildew pathogen.

### Example 3: Evaluation of the Efficacy of the Compositions According to the Invention Against Grapevine Downy Mildew

To assess the composition according to the invention efficacy against grapevine downy mildew (Plasmopara viticola), a total of 30 field trials were carried out between 2018 and 2020, in France, Italy, Spain and Greece. The product was tested on several representative grape cultivars and under different agro-climatic conditions occurring in grapevine cultivation regions in Europe. All trials were GEP (Good Experiment Practice) trials performed according EPPO PP 1/031 and French CEB M007 guidelines.

The composition prepared according to example 1 was tested at 1 L/10000 m² LWA (Leaf Wall Area) and compared with a standard elicitor (COS-OGA at 25 g/ha). The association C1 at 1 L/10000 m² LWA and copper-based fungicide at 200 g/ha was compared with a copper-based fungicide at 750 g/ha, to reduce copper quantity required to control downy mildew. The copper-based fungicide composition is a commercial solution of copper hydroxide marketed under the brand name Heliocuivre® by Action Pin.

There were 8-10 applications with intervals of 7-10 days, from BBCH 13 (3rd leaves unfolded) to BBCH 81 (beginning of ripening) crop stages so from April to August beginning. Spray volume ranged from 150 to 1000 L/ha, depending of vegetation volume and local practices. Trial design consisted of Randomized Complete Block design with 4 replicates. Plot size ranged from 4 to 12 vine-stocks (12.0-36.0 m²). To insure a sufficient disease level, artificial inoculations were performed, spraying spores suspensions on vine-stocks.

During the trial, 2-3 disease symptom observations were performed relating to downy mildew development and disease pressure. A sample of 100 leaves or 100 bunches per plot was observed. Disease severity on leaves or on bunches was recorded at each observation date. Efficacy was calculated using Abbott's formula.

Statistical analysis consisted of ANOVA with 5% alpha-risk, and Newman-Keuls test for mean comparison.

ANOVA statistical groups are represented by letters “a”, “b” and “c” in the tables below.

Data with the same letter are statistically identical. Data with a different letter are statistically different.

### Example 3a: Evaluation of the Application of a Composition Comprising Wood Rosin Alone According to the Invention on Grapevine Leaves and Bunches

TABLE 2
Results of application of the composition
comprising wood rosin on disease leaves.

| | | | Composition | |
|---|---|---|---|---|
| | | | C1 1 L/10000 | COS-OGA |
| Disease level | Number | | m² LWA | 25 g/ha |
| (disease severity | of | | (mean | (mean |
| on leaves) | trials | Untreated | efficacy) | efficacy) |
| Low (5-10% | 9 | 7.0 a | 3.4 b | 3.9 b |
| severity) | | | (51.4%) | (44.3%) |
| Moderate (10-30% | 9 | 18.1 a | 9.3 b | 10.3 b |
| severity) | | | (48.6%) | (43.1%) |
| Medium (30-50% | 2 | 36.3 a | 30.4 a | 29.4 a |
| severity) | | | (16.3%) | (19.0%) |
| High (>50% | 1 | 64.1 | 47.3 | 48.1 |
| severity) | | | (26.2%) | (25.0%) |
| All trials | 21 | 17.2 a | 10.6 b | 11.2 b |
| | | | (38.4%) | (34.9%) |

TABLE 3
Results of application of composition C1 on disease bunches.

| | | | Composition | |
|---|---|---|---|---|
| | | | C1 1 L/10000 | COS-OGA |
| Disease level | Number | | m² LWA | 25 g/ha |
| (disease severity | of | | (mean | (mean |
| on leaves) | trials | Untreated | efficacy) | efficacy) |
| Low (5-10% | 4 | 6.0 a | 3.2 b | 3.2 b |
| severity) | | | (46.7%) | (46.7%) |
| Moderate (10-30% | 3 | 16.6 a | 11.0 b | 11.6 b |
| severity) | | | (33.7%) | (30.1%) |
| Medium (30-50% | 10 | 37.9 a | 19.2 b | 26.1 b |
| severity) | | | (49.3%) | (31.1%) |
| High (>50% | 5 | 75.7 a | 62.2 b | 63.2 b |
| severity) | | | (17.8%) | (16.5%) |
| All trials | 22 | 37.8 a | 24.9 b | 28.4 b |
| | | | (34.1%) | (24.9%) |

Wood rosin-based composition C1 according to the invention shows a similar efficiency to protect both leaves and bunches than COS-OGA standard elicitor. Pleasingly, this concentration does not induce any phytotoxic effect.

### Example 3b: Evaluation of the Spray Application of a Composition Comprising Wood Rosin in Association with a Copper-Based Composition According to the Invention on Grapevine Leaves and Bunches

The comparison of the application of composition C1 in association with a copper-based composition at reduced rate with a copper-based composition at full rate is summarized in the following tables.

TABLE 4
Results of application of the composition C1 in association with
a copper-based composition at reduced rate on disease leaves.

| | | | Composition C1 | |
|---|---|---|---|---|
| | | | (1 L/10000 m² | Copper |
| Disease level | Number | | LWA) + Copper | (750 g/ha) |
| (disease severity | of | | (200 g/ha) | (mean |
| on leaves) | trials | Untreated | (mean efficacy) | efficacy) |
| Low (5-10% | 9 | 7.0 a | 2.2 b | 1.3 b |
| severity) | | | (68.6%) | (81.4%) |
| Moderate (10-30% | 7 | 16.1 a | 3.6 b | 1.1 b |
| severity) | | | (77.6%) | (93.2%) |
| Medium (30-50% | 2 | 36.3 a | 10.1 b | 7.5 b |
| severity) | | | (72.2%) | (79.3%) |
| High (>50% | 0 | — | — | — |
| severity) | | | | |
| All trials | 18 | 13.8 a | 3.6 b | 1.9 b |
| | | | (73.9%) | (86.2%) |

TABLE 5
Results of application of the composition C1 in association with
a copper-based composition at reduced rate on disease bunches.

| | | | Composition C1 | |
|---|---|---|---|---|
| | | | (1 L/10000 m² | Copper |
| Disease level | Number | | LWA) + Copper | (750 g/ha) |
| (disease severity | of | | (200 g/ha) | (mean |
| on leaves) | trials | Untreated | (mean efficacy) | efficacy) |
| Low (5-10% | 4 | 6.0 a | 1.0 b | 0.8 b |
| severity) | | | (83.3%) | (86.7%) |
| Moderate (10-30% | 3 | 16.6 a | 4.4 b | 3.3 b |
| severity) | | | (73.5%) | (80.1%) |
| Medium (30-50% | 9 | 38.0 a | 14.5 b | 7.6 c |
| severity) | | | (61.8%) | (80.0%) |
| High (>50% | 1 | 97.8 | 87.1 | 66.2 |
| severity) | | | (10.9%) | (32.3%) |
| All trials | 17 | 30.2 a | 13.8 b | 8.7 c |
| | | | (54.3%) | (71.2%) |

Spraying of composition C1 together with the copper-based composition at a reduced rate is as efficient as spraying of the copper-based composition at full rate to protect both grapevine leaves and bunches.

Furthermore, this concentration does not induce any phytotoxic effect.

Therefore wood-rosin based compositions allow reducing the use of copper while maintaining a level of efficiency to prevent downy mildew.

1. A method of using resin acids, the method comprising using the resin acids as defense gene inducers for biotic stress in a plant.

2. The method of use of resin acids according to claim 1, wherein the resin acids are derived from rosin or comprised in rosin.

3. The method of use of resin acids according to claim 2, wherein the rosin is selected from the group consisting of wood rosin, gum rosin, tall oil resin, derivatives thereof or a mixture thereof.

4. The method of use of resin acids according to claim 1, wherein the resin acids are provided as a phytosanitary product.

5. The method of use of resin acids according to claim 2, wherein the rosin derives from conifers.

6. The method of use of resin acids according to claim 3, wherein the wood rosin is extracted from pine stump or the rosin is a dismutated gum rosin or a dimerized gum rosin.

7. A composition comprising rosin and a solvent.

8. The composition according to claim 7, wherein the rosin comprises from 15 to 95 wt. % of resin acids with respect to the total weight of rosin.

9. The composition according to claim 7, further comprising an emulsifying agent, a penetrating agent and a wetting agent.

10. The composition according to claim 7, wherein the composition comprises from 15 to 25 wt. % of rosin with respect to the total weight of the composition.

11. A method of using the composition according to claim 7, the method comprising using the composition as a defense gene inducer for biotic stress in a plant.

12. A plant protection composition comprising the composition according to claim 7 and a fungicide.

13. The plant protection composition according to claim 12, wherein the fungicide is selected from the group consisting of a copper-based product, copper hydroxide, Bordeaux mixture, dithiocarbamate, dicarboximide, potassium phosphonate, dipotassium phosphonate, monopotassium phosphite, disodium phosphonate, carboxylic acid amide, cyanoimidazole, an acetamide, a phenylamide and a benzamide, phthalimide, cyanoacetamide oxime, sulfamoyl triazole, triazolo pyrimidylamine and piperidinyl triazole isoxazolines.

14. A method for treating or preventing a disease caused by at least one fungus and/or oomycete in a plant, said method comprising a step (i) of applying to the plant the composition according to claim 7.

15. The method according to claim 14, further comprising a step (ii) of applying a fungicidal composition to the plant.

16. The method of use of resin acids according to claim 2, wherein the rosin is selected from the group consisting of wood rosin, gum rosin, and derivatives thereof.

17. The composition according to claim 9, wherein the emulsifying agent is selected from the group consisting of ethoxylated castor oil, ethoxylated fatty alcohols, alkyl polyglucosides, ethoxylated rapeseed oil, glycereth-2-cocoate, polyethoxylated polyol ester, and polyoxyethylene sorbitan monolaurate.

18. The composition according to claim 9, wherein the penetrating agent is selected from the group consisting of tall oil fatty acids, unsaturated fatty acids, oleic acid, linoleic acid, saturated fatty acids, palmitic acid, and stearic acid.

19. The composition according to claim 9, wherein the wetting agent is a terpene alcohol.

20. The composition according to claim 9, wherein the wetting agent is a terpene alcohol selected from the group consisting of 2,6-dimethyloct-7-en-2-ol, alpha terpineol, 4(8)-p-menthen-1-ol, cis-3,7-dimethyl-2,6-octadien-8-ol, trans-3,7-dimethyl-2,6-octadien-1-ol, 10-hydroxymethylene-2-pinene, and 3,7-dimethyloct-6-en-1-ol.
